(19) United States
(12) Patent Application Publication
(10) Pub. No.: US 2023/0187041 A1
(43) Pub. Date: Jun. 15, 2023

(54) REAL-TIME METHOD OF BIO BIG DATA AUTOMATIC COLLECTION FOR PERSONALIZED LIFESPAN PREDICTION

(71) Applicant: INTIME BIOTECH LLC, Wilmington, DE (US)

(72) Inventor: Oleg TETERIN, Moscow (RU)

(21) Appl. No.: 17/923,678

(22) PCT Filed: May 8, 2021
(86) PCT No.: PCT/IB2021/053929

§ 371 Date: Nov. 7, 2022

Related U.S. Application Data
(60) Provisional application No. 63/022,489, filed on May 9, 2020.
(60) Provisional application No. 63/022,010, filed on May 8, 2020.

(51) Int. Cl. G16H 15/00; G16H 20/00; G16H 50/20; G16H 50/30

(52) U.S. Cl. CPC G16H 15/00; G16H 20/00; G16H 50/20; G16H 50/30; G06N 3/08

(57) ABSTRACT

Real-time method of bio big data automatic collection for personalized lifespan prediction & in-time severe diseases prevention with AI reporting system. Particularly, the present system includes at least one wearable and invention of one multi biomaterial portable container. In operation, the present system tracks in real-time vital biodata of 400+ parameters.

### FIELD OF THE INVENTION

Embodiments of the present invention pertains to system, method, devices and apparatus to automate big data collection for personalized lifespan prediction, In particular, for system, method, devices and apparatus for in-time severe diseases prevention with Artificial Intelligence (AI) reporting modules and evaluate life expectancy and general health of an individual.

### BACKGROUND OF INVENTION

Today the world is truly global in the Industry 4.0 era. Due to lifestyle changes premature death of individuals is being reported on a global scale. According to statistics, it has been reported that many individuals die early almost 30+ years prior to the body’s & organ’s physical death. Premature death is caused by severe diseases being not diagnosed and treated in-time.

Generally, Doctors avoid making a wider list of tests. Doctors do wider screenings only if the standard set of tests show not normal values. Due to lifestyle changes numerous early stage diseases need permanent wider screenings. It is a known fact that most sudden & early deaths come because of the stealth mode of the course of the disease which can’t be tracked with a standard set of tests available today.

Currently, there is a lack of detection methods & systems of early-stage diseases which can cause early death. Moreover, there is a lack of personalized approach to each patient, since the general approach is not efficient for an individual case. It has been observed that there is a low number of personal medical methods. Many times, due to misguided opinion, diagnosis is not accurately predicted by the doctor. Chances of risk of death has increased to 30% due to incorrect diagnosis, wrongly prescribed medicines & wrongly transcript by drug store workers.

Moreover, in the current innovation scenario there is a lack of distant early stage disease diagnosis solutions and systems in place. Further, due to cost constraints fewer doctors use mass spectrometer equipment to check blood & other biomaterial. In the current scheme, there remains a fear of being infected with coronavirus & other severe viruses in the hospitals & labs.

In view of the foregoing, there remains a need in the art to develop novel systems and methods. Thus, there remains a need in the art for systems, health monitoring modules where the data can be analysed using artificial intelligence (AI) algorithms, neural networks and the like.

### SUMMARY OF THE INVENTION

Embodiments of the present disclosure relate to systems, health monitoring modules where the data is analysed using artificial intelligence (AI) algorithm, neural networks and the like. Particularly, the present system and method provides one or more humans with at least one or more years of active life. In operation, invention is provided with at least one wearable or connection to health data aggregation platform, such as HealthKit for Apple gadgets or/and GoogleFit for Android gadgets or/and Samsung Health application and the like and invention of one multi biomaterial portable container for remote biomaterial collection or/and collecting biomaterial at the users home or another location with the nurse or in the laboratory or the outcomes of the biomaterial tests could be entered manually. In cases of remote biomaterial collection and further analysis, the outcome data is retrieved by the lab directly to the company’s server or the outcome could be manually input by the user. All together they track in real-time vital biodata of 400+ bio parameters. Invention provides a hardware and a software system which permanently analyses this data and brings it to user’s attention in a mobile application in the form of disease risk report.

In accordance with various embodiments of the present invention, the present system is AI trained on 70+ years of human’s clinical trials. Multiple modules of the present invention retrieve data from structured & unstructured medical databases. The retrieved Big Data provides an up to 100% bio parameters match between the user’s bio parameters and bio parameters from medical databases. Subsequently, the present system generates a personal machine learning algorithm which publishes personal recommendations reports on how to lower disease risk. Users can extend their active life if they consider risk and follow recommendations.

In accordance with various embodiments of the present invention, a prediction system to assess life expectancy and a plurality of health parameter factors are disclosed. In use, the prediction system includes a monitoring module configured to monitor the plurality of health parameter factors, an assessment module configured as a neural network trained on data retrieved from a first database storing the plurality of health parameter factors from many individuals, an evaluation module configured to evaluate human data training sample to draw conclusions based on a data set of a large number of people and summarizing at least one characteristic from the human data training sample, a second stage module configured to develop a trained neural network and said trained neural network is a network that analyses a plurality of historical data of an individual, a generation module configured to provide output data and the output data is a human health assessment factor that is directly related to life expectancy of an individual. Particularly, the human data training sample is selected from a plurality of input parameters obtained from at least one medical record, or/and at least one wearable device worn by an individual or/and 1 health data aggregation platform like HealthKit or GoogleFit or other health application, surveys, questionnaires, manual input and the multiple input parameters are stored in the first database, and the trained neural network take into account a plurality of time-periods of life that affect both positively and negatively prognosis of life expectancy of the individual.

In accordance with various embodiments of the present invention, a method for predicting and assessing life expectancy is disclosed. The method includes the steps of monitoring a plurality of health parameter factors and assessing a neural network trained on data retrieved from a first database storing the plurality of health parameter factors from many individuals, evaluating human data training sample to draw conclusions based on a data set of a large number of people and summarizing at least one characteristic from the human data training sample, developing a trained neural network and the trained neural network is a network that analyzes a plurality of historical data of an individual, and, generating output data and the output data is a human health assessment factor that is directly related to life expectancy of the exact individual based on his historical data. Particularly, the human data training sample are selected from a plurality of input parameters obtained from at least one medical record or/and at least one wearable device worn by an individual or/and 1 health data aggregation platform like HealthKit or GoogleFit or other health application, surveys, questionnaires, manual input and the like and the plurality of input parameters are stored in the first database.

In one embodiment, trained neural network take into account a plurality of time-periods of life that affect both positively and negatively prognosis of life expectancy of the individual.

In accordance with various embodiments of the present invention, the present method further includes the steps of retrieving a list of required parameters for permanent tracking of at least one or more upcoming diseases, which can shorten life dramatically of the individual, structuring received personal datasets of multiple individuals and forming an each user digital profile based on matches received from at least one user personal dataset (various bio parameters) with multiple datasets (with the same name list of bio parameters) of general population received from multiple databases by artificial Intelligence (AI) engine module, providing personal severe diseases prevention recommendations to the individual and generating a personalized disease risk report.

In one embodiment, the list of required parameters are real-time vital biodata of individuals and artificial Intelligence (AI) engine module is configured to extract needed data from unstructured data.

In accordance with various embodiments of the present invention, the evaluating human data training sample step further includes the steps of instantly evaluating human data obtained at a particular point in time wherein a data set is of a large number of people, and summarizing a plurality of characteristics of people divided in groups with same characteristics in each group (segmented by the same age or/and same gender or/and same residency or/and same genetic disposition or/and same environment or/and same behavioral patterns and other same parameters) from the training sample, developing a historical data neural network that analyzes historical data of said individual & segmented by the same parameters group of individuals and wherein said historical data neural network is trained with a large amount of data over a long period of time by selecting at least one architecture of a neural network, and recognizing a large number of patterns from a plurality of input parameters to evaluate individual relationship between a plurality of person’s life and their respective health level.

In one embodiment, at least one or in combination of a personalized network parameters are selected from a plurality of genetic characteristics, current physical condition of the body, blood parameters, environment influence, behavioral patterns, nutrition, and psycho emotional state of a person to determine accurate forecast for said individual.

### BRIEF DESCRIPTION OF THE DRAWINGS

So that the manner in which the above recited features of the present invention is to be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a block diagram of a prediction system to assess life expectancy and multiple health parameter factors, according to an example embodiment;

FIG. 2 is a block diagram of an evaluation module and other sub-modules, according to one or more embodiments of the present invention;

FIG. 3 is a block diagram of an example computing system structured to perform assess life expectancy operations, according to an example embodiment; and

FIG. 4 illustrates a flowchart of a method for assessing life expectancy, according to one or more embodiments of the present invention;

### DETAILED DESCRIPTION

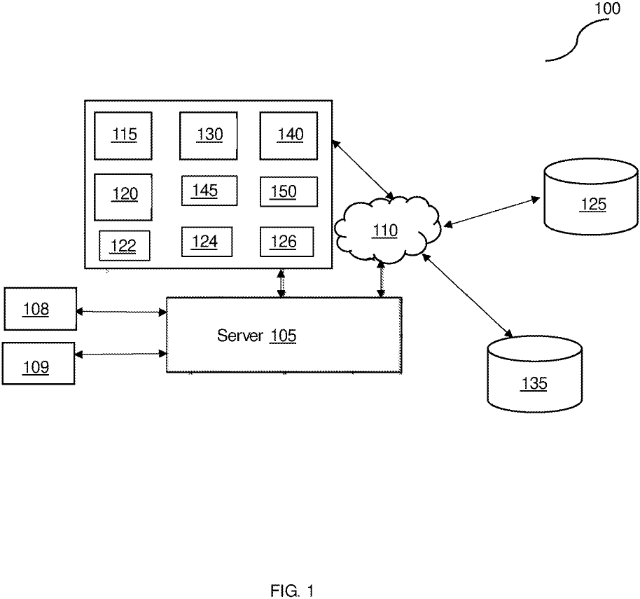

The present invention relates to systems, health monitoring modules where the data can be analysed using artificial intelligence (AI) algorithms and the like. The principle of the present invention and their advantages are best understood by referring to FIG. 1 to FIG. 4. In the following detailed description of illustrative or exemplary embodiments of the disclosure, specific embodiments in which the disclosure may be practiced are described in sufficient detail to enable those skilled in the art to practice the disclosed embodiments.

The following detailed description is, therefore, not to be taken in a limiting sense, and the scope of the present disclosure is defined by the appended claims and equivalents thereof. References within the specification to “one embodiment,” “an embodiment,” “embodiments,” or “one or more embodiments” are intended to indicate that a particular feature, structure, or characteristic described in connection with the embodiment is included in at least one embodiment of the present disclosure. The word “dataset” is interchangeably used with “bio parameters”. Dataset refers to matching of the bio-parameters.

Various embodiments of the present invention provide methods, systems, health monitoring modules where the data can be analysed using artificial intelligence (AI) algorithms and the like.

Systems, methods, and computer-readable media of the present disclosure may utilize artificial intelligence (AI) and, more specifically, machine learning (ML). Some of the ways in which AI and ML are contributing in the monitoring multiple health parameters include real-time insights into health and performance of a technology stack. Additionally, AI may be implemented in a recommender module that suggests recommendation steps based on past similar incidents from general population or/and from segmented groups with same parameters or/and from said individual and performs self-healing through automation for recurring incidents. Additionally, AI may be used to correlate anomalies to create unique situations and identifies potential cause and impact for anomalies. AI modules is able to learn how to act and what to recommend to do, based on learning patterns of the past cases.

With reference now to the Figs, particularly like reference numbers denote parts and different components of the present system.

FIG. 1 illustrates a block diagram of a prediction system 100 to assess life expectancy and multiple health parameter factors, according to an example embodiment. In use, the present system 100 is configured to evaluate life expectancy and general health of a person based on as many factors of his life as possible.

In one embodiment, one or more factors are selected from height, weight, age, gender, nutrition, physical activity level, nature of work, at least one geographical location, nationality, environmental conditions, stress level, genetic characteristics, diseases and many other factors. Particularly, the one or more geographical locations are selected from past location of the individual and the present location of the individual.

The prediction system 100 includes a user interface 109, a monitoring module 115 configured to monitor multiple health parameter factors, and an assessment module 120 configured as a neural network trained on data retrieved from a first database 125 storing the multiple health parameter factors from many individuals. Particularly, the network input data is a set of parameters obtained from medical records, wearable devices, questionnaires and other sources. Moreover, the output of the network is a human health factor that is directly related to life expectancy. The higher the factor, the longer and better the life of a person will last.

In use, the server 105 sends a request to AI engine module 130 to generate the list of required parameters for permanent tracking the upcoming diseases, which can shorten life dramatically. More than 400+ parameters can be tracked and acted upon for better living. These parameters are identified and retrieved from numerous medical databases based on the present structuring unstructured approach modules. The list of those required parameters are stored in the Big Data server 105 collected from medical database 135 and not limited to other scientifical articles which output approved by World Health Organization or/and World medical Association or/and World Federation of Public Health Organization & other like associations. The list of parameters associated with exact diseases can vary depending on the new World health Organization & other associations decisions. The term “Server” and “Big Data Server” are used interchangeably in the present invention.

Furthermore, the first database 125 and the medical database 135 may be a relational database, which is a database with a structure that recognises relationships among stored items. Preferably, the first database 125 and the medical database 135 are connected to the user interface 109 to provide advanced searching capabilities that allow users to search for information in the databases 125, 135 via the user interface 109. It is also preferable for the data obtained to be viewed by the user via the user interface 109 in different methods such as plots, charts, tables, and graphs. Geographical locations (past, present), epidemic networks, pandemic networks and other data may also be plotted on a map to give a general view of the cases. Additionally, a pivot table may be constructed to allow the users to sort and filter the data.

In one embodiment, the system 100 further includes an evaluation module 140, a second stage module 145, and a generation module 150. Particularly, the evaluation module 140 is configured to evaluate human data training sample to draw conclusions based on a data set of a large number of people and summarizing at least one characteristic from the human data training sample. Further, the second stage module 145 is configured to develop a trained neural network and the trained neural network is a network that analyses multiple historical data of an individual.

In one embodiment, the generation module 150 is configured to provide output data and the output data is a human health assessment factor that is directly related to life expectancy of an individual. Particularly, the human health assessment factor is a combination of values obtained from test data of the individual and group of individuals with same parameters and at least one functional characteristic of each individual body.

In use, the human data training sample is selected from a plurality of input parameters obtained from at least one medical record, at least one wearable device worn by an individual, surveys, questionnaires and the like and multiple input parameters are stored in the first database 125, and the trained neural network take into account a plurality of time-periods of life that affect both positively and negatively prognosis of life expectancy of the individual.

Generally, to organize process of data collection and network training, it is necessary to solve many technical and organizational problems. Among the technical ones are the task of forming a training sample and the target value of life expectancy or human health factor. In use, the present system 100 includes steps to formulate a criterion for assessing their health factor for existing patient data sets. Such a health factor is a synthetic measure, depending on the number of systemic diseases, the current values of the tests and the functional characteristics of the body.

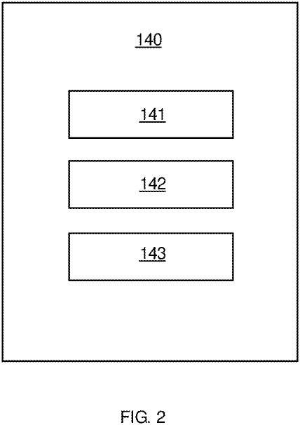

FIG. 2 is a block diagram of an evaluation module 140 and other sub-modules, according to one or more embodiments of the present invention. Particularly, the evaluation module 140 further includes a first stage evaluation sub-module 141, a second stage evaluation sub-module 142, and a third stage evaluation sub-module 143. In use, the first stage evaluation sub-module 141 is configured to instantly evaluate human data obtained at a particular point in time wherein a data set is of a large number of people, and summarizing a plurality of characteristics of people from the training sample. The second stage evaluation sub-module 142 is configured to develop a historical data neural network that analyses historical data of each individual and segmented by the same parameters group of individuals and the historical data neural network is trained with a large amount of data over a long period of time by selecting at least one architecture of a neural network. The instant data retrieved is not able to provide information about changes in human health throughout lifespan. The current analysis slice at the time of illness provides an incorrect biased estimate. A qualitative analysis requires a network that evaluates the time series of a person’s testimony over a lifetime. The present system is enabled to train neural network to take into account periods of life that affect both positively and negatively the prognosis of life expectancy. For example, based on information taken by wearable devices that a person has been involved in sports throughout his life, he will be able to predict the maximum values of human health in old age and long-life spans. Data on living in an environmentally disadvantaged area with low life expectancy will lower overall health scores. To train such a neural network, it is necessary to accumulate a large amount of data over a long period of time, correctly choose the architecture of the neural network.

In one embodiment of the present invention, the one or more architecture of a neural network is selected from a recurrent neural network and convolutional neural network.

The third stage evaluation sub-module 143 is configured to recognize a large number of patterns from a plurality of input parameters to evaluate individual relationship between a plurality of person’s life and their respective health level. Further, the third stage evaluation sub-module 143 is further configured to form a core network. The core network is trained on a large data sample and subsequently the core network is adjusted for a specific individual by training the core network on the specific individual for a significant period of time.

In one embodiment, the third stage evaluation sub-module 143 is the most personalized network where the network parameters will take into account the individual relationship between the parameters of a person’s life and their health level. This is affected by genetic characteristics, the current physical condition of the body, blood parameters, nutrition, and the psycho-emotional state of a person. The more such factors are taken into account, the more accurate a forecast can be made. Such a set will also be able to suggest that a person is in conditions uncomfortable for his body. For example, a trained person to run 5 kilometres will only benefit, and a person with signs of heart disease can lead to death. A neural network of the third level should recognize a large number of patterns of input parameters, be able to accumulate information about a particular person, i.e., be recursive.

In one embodiment, the one or in combination of a personalized network parameters are selected from multiple genetic characteristics, current physical condition of the body, blood parameters, nutrition, and psycho emotional state of a person to determine accurate forecast for the individual.

In one embodiment, the trained neural network is trained using one or more algorithms including but not limited to stochastic gradient descent optimizer, adaptive moment estimation optimization, root mean square propagation optimization, linear Regression, logistic regression, monte carlo method, markov models (including Markov chain, Hidden Markov model, Markov decision process, Partially observable Markov decision process), Transformer (NN), Support Vector Machines, Linear SVC, k-Nearest Neighbors algorithm, Naive Bayes , Perceptron, Decision Tree Classifier, Random Forests, XGB Classifier, LGBM Classifier, Gradient Boosting Classifier, Ridge Classifier, Bagging Classifier, ensembles of models/approaches, based on described points.

In one embodiment of the present invention, parameters are being split in two categories for online and offline permanent tracking in approximate proportion 50+ parameters for online tracking, including manual input and 350+ for offline, including manual input. However, the number of parameters can be different depending on individual person lifestyle and environment.

In one embodiment of the present invention, the prediction system 100 further incudes the server 105 configured to send a request to an artificial Intelligence (AI) engine module 122 to retrieve a list of required parameters for permanent tracking of at least one or more upcoming diseases, which can shorten life dramatically of the individual. Particularly, the artificial Intelligence (AI) engine module 122 configured to structure received personal datasets of multiple individuals and form a user digital profile based on matches received from at least one user personal dataset with multiple datasets of general population received from multiple databases. The prediction system 100 further incudes a recommendation module 124 configured to provide personal severe diseases prevention recommendations; and a report module 126 configured to generate a disease risk report; wherein said list of required parameters are real-time vital biodata of individuals and wherein artificial Intelligence (AI) engine module 122 is configured to extract needed data from unstructured data.

In one embodiment, every time data retrieved from the user is different as compared previous stored data, new recommendation report is generated for the user to review.

In one embodiment of the present invention, the list of required parameters are split into an offline permanent tracking category and an online permanent tracking category.

In one embodiment of the present invention, the prediction system 100 further includes at least one smart wearable 108 (or aggregation platform such as HealthKit, GoogleFit or other health mobile applications) which contains at least one sensor to record physical properties, include anyone or combination of blood pressure on both hands (morning and night), heart rate variability, resting heart rate, VO2max (direct measurement or Cooper test score), manual input of waist, hip, neck, wrist circumferences, common diseases (incl. depression, anxiety, cyberchondria, etc.), prescribed medications, nutritional supplements, entheogenic, recreational, performance enhancing and other medicines, movement data and sleep mode, mood and mental performance self-esteem, libido, body temperature, lung function, blood glucose, various active motion tests, outside temperature, humidity level, illumination level, electromagnetic fields, ionizing radiation & others from the body online and other physical and biodata, and at least one interface with the network 110 capable of utilizing the information obtained from the at least one sensor. All data is placed through the communication network 110 on the server 105.

In one embodiment of the present invention, the list of required parameters includes age, sex, height, nationality, thigh/neck circumferences, Raffier-Dickson index for measuring aerobic endurance, reaction time test results, hand strength, Strange and Genchi tests, high frequency auditory test, visual acuity check orthostatic blood pressure restoration test, ECG, EEG, Pwv, hands-Free test, breath holding time after deep exhalation, and flexibility tests.

In one embodiment of the present invention, the mobile application distantly collects personal bio data with 50+ parameters (datasets) via existing wearable devices 108.

In another embodiment of the present invention, the portable container (not shown) distantly collects personal bio data of 350+ parameters, incl. blood omics profile, urine & faeces profile, nail, hair, skin data, forming another dataset. All this data is collected from the body offline thru “all in one” Rapid Diagnostics Test (RDT) device portable container or collected by the nurse, or in the lab or manually input.

In yet another embodiment of the present invention, the biomaterial biodata (datasets) from portable container device is delivered to remote location by any available service. Particularly, the remote location is closest mass spectrometer laboratory or another lab. These datasets are analysed with mass spectrometer & other laboratory equipment to determine user values in over 350+ parameters.

In yet another embodiment of the present invention, 350+ parameters data are being sent via the internet from a mass spectrometer laboratory to the company’s server 105 for future AI & ML automatic calculations.

In yet another embodiment of the present invention, providing one or more parameters which currently can’t be tracked online, including blood omics & other biomaterial parameters, Bondarevsky test & other tests which needs the third-party participation for wrist, neck, hips, waist & etc measurements could be tracked online by the development of new technologies.

In yet another embodiment of the present invention, some parameters such as balancing test on one leg (Bondarevsky test) & others which currently can’t be tracked automatically online are being entered manually with a third person help until trackers can measure them distantly. In operation, the collected personal bio data is stored in the server 105.

In yet another embodiment of the present invention, AI engine module 122 executes a set of instructions to enable AI algorithm for extracting needed data in the structured way (structured data) from unstructured way (unstructured data). Further, the AI engine module 122 extracts data only from reputable medical databases such as PubMed, Mimic & others 10. For example, medical bases: https://www.medscape.com/viewarticle/451577 3. Particularly, all extracted data from medical bases datasets 10 are based only on human clinically proven trials reports. These reports were given to the databases by researches, scientists, professional literature, encyclopaedias & etc and priorly approved by World Health Organization & other like associations.

In yet another embodiment of the present invention, AI engine module 122 extracts from medical databases datasets based on the same (50+ online & 350+ offline) parameters that are tracked distantly from the user’s body. In operation, the AI algorithm matches received user’s personal datasets with datasets of general population received from TOP 10+ medical databases 10 such as PubMed, Mimic & others.

In yet another embodiment of the present invention, the summary of that personally adjusted data is shown in a clear and understandable, even to 10+ year old, way and tells what these results mean to the user and what to do with that collected data to improve own health and avoid potential diseases and early death. There is distant access (with user’s consent) to mobile application of the user’s existing treating doctor.

In yet another embodiment of the present invention, no matter how often, every time when data from a user is changed, described processes are being held from the very beginning, bringing new results & recommendations in a new report. In operation, the server 105 scans the databases 125,135 each day for having more human clinical trials reports being published. Every day a new report is being generated and provided to the server 105 for updating all previous AI engine calculations incl. All risk and recommendation reports, if new relevant data arrived.

In yet another embodiment of the present invention, company contracts medical institutions and permanently obtains personalized or depersonalized biodata of their users. The Big Data server 105 structures personalized or depersonalized biodata in to social, demographical, national, sex, age and other parameters modules. Each module is configured for linking with exact persons digital profile with maximum match for higher report rate. This is done to improve a company’s algorithms in order to raise the accuracy of risk level calculations report rate.

In yet another embodiment of the present invention, the AI engine module 130 is configured to develop personalized algorithms for each user’s report. The number of different reports could be as many as many changings in at least one parameter of any dataset.

In yet another embodiment of the present invention, after 3 to 5+ years of company’s users tracking history, when the server 105 receive sufficient exact user’s personal data, the AI engine module 130 start using collected personal data to generate new AI algorithms for user’s personal diseases risk prediction & ML algorithm generate personal prevention calculations (report) without usage of general populations datasets forming Big Data of the exact users 400+ parameters digital profiles, forming a “new way” of measurements & predictions. The “old way” will be only for new users without online tracking 3 to 5+ years history.

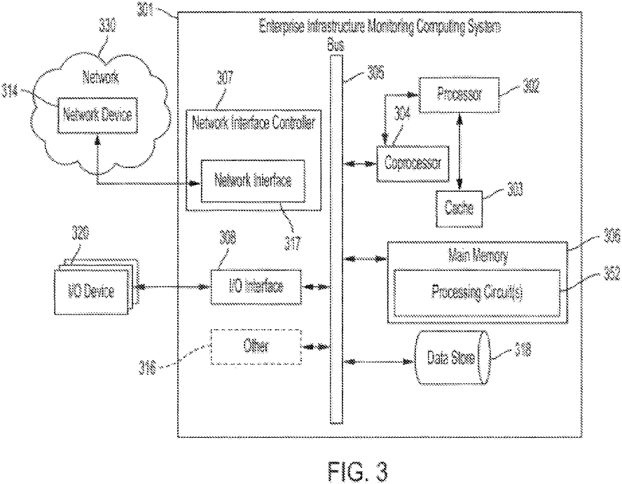

Referring now to FIG. 3, a block diagram 300 of an example prediction system 301 is shown to perform assess life expectancy operations, according to an example embodiment. The prediction system 301 is suitable for use in implementing the computerized components described herein, in accordance with an illustrative implementation. In broad overview, the prediction system 301 includes a processor 302 for performing actions in accordance with instructions, e.g., instructions held in cache memory 303. The illustrated example prediction system 301 includes one or more processors 302 and coprocessors 304 in communication, via a bus 305, with main memory 306 comprising computer-executable code embodying the processing circuit 352, a network interface controller 307, an input/output (I/O) interface 308, and a data store 318, etc. In some implementations, the prediction system 301 may include additional interfaces or other components 316.

As shown, the main memory 306 includes the processing circuit 352, which may be structured to perform the functions described in relation to FIG. 1 and FIG. 2. One of skill will appreciate that various arrangements suitable for practicing the principles disclosed herein are within the scope of the present disclosure.

In some implementations, a processor 302 can be configured to load instructions from the main memory 306 (or from data storage) into cache memory 303. Furthermore, the processor 302 can be configured to load instructions from cache memory 303 into onboard registers and execute instructions from the onboard registers. In some implementations, instructions are encoded in and read from a read-only memory (ROM) or from a firmware memory chip (e.g., storing instructions for a Basic I/O System (BIOS)), not shown.

The network interface controller 307 can be configured to control one or more network interfaces 317 for connection to network devices 314 (e.g., for access to a network 330). The I/O interface 308 can be configured to facilitate sending and receiving data to various I/O devices 320 such as, but not limited to, keyboards, touch screens, microphones, motion sensors, video displays, speakers, haptic feedback devices, printers, and so forth. In some implementations, one or more of the I/O devices 320 are integrated into the prediction system 301. In some implementations, one or more of the I/O devices 320 are external to, and separable from, the prediction system 301.

Still referring to FIG. 3, the bus 305 is an interface that provides for data exchange between the various internal components of the prediction system 301, e.g., connecting the processor 302 to the main memory 306, the network interface controller 307, the I/O interface 308, and data store 318. In some implementations, the bus 305 further provides for data exchange with one or more components external to the prediction system 301, e.g., other components 316. In some implementations, the bus 305 includes serial and/or parallel communication links. In some implementations, the bus 305 implements a data bus standard such as integrated drive electronics (IDE), peripheral component interconnect express (PCI), small computer system interface (SCSI), or universal serial bus (USB). In some implementations, the prediction system 301 has multiple busses 305.

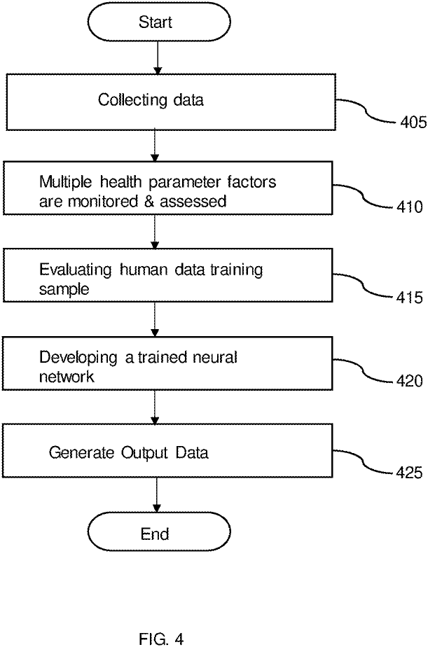

Reference is now made to FIG. 4, wherein FIG. 4 illustrates a flowchart of a method for predicting and assessing life expectancy, according to one or more embodiments of the present invention.

Initially, data is collected and stored in the first database 125. In use, the first database 125 stores the multiple health parameter factors from many individuals in step 405. The first database 125 include anyone or combination of data including blood pressure on both hands (morning and night), heart rate variability, resting heart rate, VO2max (direct measurement or Cooper test score), waist circumferences, common diseases (incl. depression, anxiety, cyberchondria, etc.), prescribed medications, nutritional supplements, entheogenic, recreational, performance enhancing and other medicines, movement data and sleep mode, mood and mental performance self-esteem, libido, body temperature, lung function, blood glucose, various active motion tests, outside temperature, humidity level, illumination level, electromagnetic fields, ionizing radiation & others from the body online and other physical and biodata. In use, data of individuals is stored individually. Further, the data is segmented based on age, sex & all other parameters. For e.g., make groups of same parameters and ailments (only male, 45 years, living in New York in metropolitan area with stroke in the past, smoking, minimum fitness activity & etc). The method proceeds to step 410.

At step 410, multiple health parameter factors are monitored and assessed by the neural network trained on data from many individuals retrieved from the first database 125 and the medical database 135. The human data training sample is selected from a plurality of input parameters obtained from at least one medical record, at least one wearable device worn by an individual, surveys, questionnaires and the like and said plurality of input parameters are stored in the first database 125.

In one embodiment, a list of required parameters for permanent tracking of at least one or more upcoming diseases, which can shorten life dramatically of the individual are retrieved. Thereon, received personal datasets of multiple individuals are structured and different user digital profiles are formed based on matches received from at least one user personal dataset with multiple datasets of general population received from multiple databases by the artificial Intelligence (AI) engine module 122.

In one embodiment, the present method includes the step of recalculating all risks & recommendations as often as at least one vital parameter significantly changes.

The method proceeds to step 415. At step 415, human data training sample is evaluated to draw conclusions based on a data set of a large number of people and summarizing at least one characteristic from the human data training sample. Particularly, the trained neural network takes into account a plurality of time-periods of life that affect both positively and negatively prognosis of life expectancy of said individual.

In one embodiment, the evaluating human data training sample step 425 further includes the steps of instantly evaluating human data obtained at a particular point in time wherein a data set is of a large number of people, and summarizing a plurality of characteristics of people from the training sample. Thereon, developing a historical data neural network that analyses historical data of the individual. In use, the historical data neural network is trained with a large amount of data over a long period of time by selecting at least one architecture of the neural network. In operation, a large number of patterns are recognised from multiple input parameters to evaluate individual relationship between a number of person’s life and their respective health level.

In one embodiment, the at least one or in combination of a personalized network parameters are selected from multiple genetic characteristics, current physical condition of the body, blood parameters, nutrition, and psycho emotional state of a person to determine accurate forecast for the individual.

In yet another embodiment, the core network is formed and the core network is trained on a large data sample and subsequently the core network is adjusted for a specific individual by training the core network on the specific individual for a significant period of time.

Thereon, developing a trained neural network and the trained neural network is a network that analyzes a plurality of historical data of an individual at step 420. Particularly, the trained neural network takes into account a plurality of time-periods of life that affect both positively and negatively prognosis of life expectancy of the individual.

Subsequently, output data is generated at step 425. Particularly, the output data is a human health assessment factor that is directly related to life expectancy of the individual. For example, the human health assessment factor is equal to longer lifespan of the individual when the evaluated risk of severe diseases is low.

In one embodiment, personal severe diseases prevention recommendations are provided to the individual and a disease risk report is generated. Particularly, the list of required parameters are real-time vital biodata of individuals and the artificial Intelligence (AI) engine module 122 is configured to extract needed data from unstructured data.

In one embodiment, the human health assessment factor is a combination of values obtained from test data of the individual and at least one functional characteristic of the individual body. Particularly, the one or more functional characteristic of the individual body is selected from glucose, cholesterol, oncology markers and the like.

Accordingly, the present invention has a number of advantages. The present system is a 4P Medicine system - Prevention, Prediction, Participatory, Personalized. The present invention goal is to catch the “butterfly effect” of each person’s life trajectory, when it is early to make necessary changes for these people so they can live 20+ active and happy years without limitations. Moreover, the present instant invention has the technical effect of providing personal and national interest solutions to everyone, each nation and region. By tracking top 20 severe diseases at the onset aka earliest known stages, the present invention is able to increase longevity of individuals and extend their lifespan. Further, early analysis saves money on expensive treatments, when severe disease is already in progress.

It is the object of the present invention, to deploy systems, health monitoring modules where the data can be analysed using artificial intelligence (AI) algorithms and the like. In other words, the data can be analysed using supervised learning, support vector network, machine learning, AI and the like. In some embodiments, algorithms and rules are used by machine learning to analyse the output of the system and provide various types of information for use in a clinical environment. For example, the data may be used for diagnoses, testing and teaching in some embodiments. Machine learning algorithms and AI backed reports in lifespan predictions are only in the beginning of its evolution.

Moreover, the present invention is monitoring more than 20+ parameters that current real-time online trackers can monitor, with health status & generating recommendation reports. There are no portable containers, which can collect distantly various types of biomaterials in one container and then to have biomaterial to be transcript for 400+ parameters, which can safely store biomaterials inside of the container & being brought in-time to the special lab.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

1. A prediction system to assess life expectancy and a plurality of health parameter factors, said prediction system comprising:
a monitoring module configured to monitor said plurality of health parameter factors;
an assessment module configured as a neural network trained on data retrieved from a first database storing said plurality of health parameter factors from many individuals;
an evaluation module configured to evaluate human data training sample to draw conclusions based on a data set of a large number of people and summarizing at least one characteristic from said human data training sample;
a second stage module configured to develop a trained neural network and said trained neural network is a network that analyzes a plurality of historical data of an individual and group of individuals with same parameters; and
a generation module configured to provide output data and said output data is a human health assessment factor that is directly related to life expectancy of an individual;
wherein said human data training sample is selected from a plurality of input parameters obtained from at least one medical record, at least one wearable device worn by an individual, surveys, questionnaires and the like and said plurality of input parameters are stored in said first database.

2. The prediction system as claimed in claim 1, wherein said trained neural network take into account a plurality of time-periods of life that affect both positively and negatively prognosis of life expectancy of said individual.

3. The prediction system as claimed in claim 1, wherein said human health assessment factor is a combination of values obtained from test data of said individual and group of individuals with same parameters and at least one functional characteristic of each individual body.

4. The prediction system as claimed in claim 1, wherein said plurality of health parameter factors are selected from at least one or in a combination from height, weight, age, gender, nutrition, physical activity level, nature of work, at least one geographical location, nationality, environmental conditions, stress level, genetic characteristics, diseases and the like of each individual.

5. The prediction system as claimed in claim 1, wherein said evaluation module further comprises:
a first stage evaluation sub-module configured to instantly evaluate human data obtained at a particular point in time wherein a data set is of a large number of people, and summarizing a plurality of characteristics of people from said training sample;
a second stage evaluation sub-module configured to develop a historical data neural network that analyzes historical data of said individual and group of individuals with similar parameters and wherein said historical data neural network is trained with a large amount of data over a long period of time by selecting at least one architecture of a neural network;
a third stage evaluation sub-module configured to recognize a large number of patterns from a plurality of input parameters to evaluate individual relationship between a plurality of person’s life and their respective health level;
wherein at least one or in combination of a personalized network parameters are selected from a plurality of genetic characteristics, current physical condition of the body, blood parameters, nutrition, and psycho emotional state of a person to determine accurate forecast for said individual.

6. The prediction system as claimed in claim 5, wherein said third stage evaluation sub-module further configured to form a core network and said core network is trained on a large data sample and subsequently said core network is adjusted for a specific individual by training said core network on said specific individual for a significant period of time.

7. The prediction system as claimed in claim 1, wherein said trained neural network is trained using one or more algorithms comprising stochastic gradient descent optimizer, adaptive moment estimation optimization and root mean square propagation optimization.

8. The prediction system as claimed in claim 1, wherein said prediction system further comprises:
a server configured to send a request to an artificial Intelligence (Al) engine module to retrieve a list of required parameters for permanent tracking of at least one or more upcoming diseases, which can shorten life dramatically of said individual and said artificial Intelligence (Al) engine module configured to structure received personal datasets of multiple individuals and form a user digital profile based on matches received from at least one user personal dataset with multiple datasets of general population received from multiple databases;
a recommendation module configured to provide personal severe diseases prevention recommendations; and
a report module configured to generate a disease risk report;
wherein said list of required parameters are real-time vital biodata of individuals and wherein artificial Intelligence (Al) engine module is configured to extract needed data from unstructured data.

9. The prediction system as claimed in claim 8, wherein said list of required parameters are split into an offline permanent tracking category and an online permanent tracking category.

10. The prediction system as claimed in claim 8, wherein said prediction system further comprises at least one smart wearable which contains at least one sensor to record physical properties, include anyone or combination of blood pressure on both hands (morning and night), heart rate variability, resting heart rate, VO2max (direct measurement or Cooper test score), waist circumferences, common diseases (incl. depression, anxiety, cyberchondria, etc.), prescribed medications, nutritional supplements, entheogenic, recreational, performance enhancing and other medicines, movement data and sleep mode, mood and mental performance self-esteem, libido, body temperature, lung function, blood glucose, various active motion tests, outside temperature, humidity level, illumination level, electromagnetic fields, ionizing radiation & others from the body online and other physical and biodata, and at least one interface with a network capable of utilizing the information obtained from said at least one sensor.

11. The prediction system as claimed in claim 8, wherein said list of required parameters comprises age, sex, height, nationality, thigh/neck circumferences, Raffier-Dickson index for measuring aerobic endurance, reaction time test results, hand strength, Strange and Genchi tests, high frequency auditory test, visual acuity check orthostatic blood pressure restoration test, ECG, EEG, Pwv, hands-Free test, breath holding time after deep exhalation, and flexibility tests.

12. A method for predicting and assessing life expectancy, said method comprising:
monitoring a plurality of health parameter factors and assessing a neural network trained on data retrieved from a first database storing said plurality of health parameter factors from many individuals;
evaluating human data training sample to draw conclusions based on a data set of a large number of people and summarizing at least one characteristic from said human data training sample;
developing a trained neural network and said trained neural network is a network that analyzes a plurality of historical data of an individual and group of individuals with same parameters; and
generating output data and said output data is a human health assessment factor that is directly related to life expectancy of each individual;
wherein said human data training sample is selected from a plurality of input parameters obtained from at least one medical record, at least one wearable device worn by an individual, surveys, questionnaires, manual input and the like and said plurality of input parameters are stored in said first database; and wherein said trained neural network take into account a plurality of time-periods of life that affect both positively and negatively prognosis of life expectancy of said individual.

13. The method as claimed in claim 12, wherein said method further comprising the steps of:
retrieving a list of required parameters for permanent tracking of at least one or more upcoming diseases, which can shorten life dramatically of said individual;
structuring received personal datasets of multiple individuals and forming a user digital profile based on matches received from at least one user personal dataset with multiple datasets of general population received from multiple databases by artificial Intelligence (Al) engine module;
providing personal severe diseases prevention recommendations to said individual and generating a disease risk report;
wherein said list of required parameters are real-time vital biodata of individuals and wherein artificial Intelligence (Al) engine module is configured to extract needed data from unstructured data.

14. The method as claimed in claim 13, wherein said evaluating human data training sample step further comprising the steps of:
instantly evaluating human data obtained at a particular point in time wherein a data set is of a large number of people, and summarizing a plurality of characteristics of people from said training sample;
developing a historical data neural network that analyzes historical data of said individual and wherein said historical data neural network is trained with a large amount of data over a long period of time by selecting at least one architecture of a neural network;
recognizing a large number of patterns from a plurality of input parameters to evaluate individual relationship between a plurality of person’s life and their respective health level;
wherein at least one or in combination of a personalized network parameters are selected from a plurality of genetic characteristics, current physical condition of the body, blood parameters, nutrition, environment, behavior and psycho emotional state of a person to determine accurate forecast for said individual.

15. The method as claimed in claim 14, wherein a core network is formed and said core network is trained on a large data sample and subsequently said core network is adjusted for a specific individual by training said core network on said specific individual for a significant period of time.

16. The method as claimed in claim 12, wherein said human health assessment factor is a combination of values obtained from test data of said individual and at least one functional characteristic of said individual body.

17. The method as claimed in claim 12, wherein said plurality of health parameter factors are selected from at least one or in a combination from height, weight, age, gender, nutrition, physical activity level, nature of work, at least one geographical location, nationality, environmental conditions, stress level, genetic characteristics, diseases and the like of each individual.

18. The method as claimed in claim 12, wherein said trained neural network is trained using one or more algorithms comprising stochastic gradient descent optimizer, adaptive moment estimation optimization and root mean square propagation optimization & other.

19. The method as claimed in claim 12, wherein said method further comprises the steps of recording physical properties of said individual include anyone or combination of blood pressure on both hands (morning and night), heart rate variability, resting heart rate, VO2max (direct measurement or Cooper test score), waist circumferences, common diseases (incl. depression, anxiety, cyberchondria, etc.), prescribed medications, nutritional supplements, entheogenic, recreational, performance enhancing and other medicines, movement data and sleep mode, mood and mental performance self-esteem, libido, body temperature, lung function, blood glucose, various active motion tests, outside temperature, humidity level, illumination level, electromagnetic fields, ionizing radiation & others from the body online and other physical and biodata, and at least one interface with a network capable of utilizing the information obtained from at least one sensor mounted on at least one smart wearable or portable.

20. The method as claimed in claim 12, wherein said list of required parameters comprises age, sex, height, nationality, thigh/neck circumferences, Raffier-Dickson index for measuring aerobic endurance, reaction time test results, hand strength, Strange and Genchi tests, high frequency auditory test, visual acuity check orthostatic blood pressure restoration test, ECG, EEG, Pwv, hands-Free test, breath holding time after deep exhalation, and flexibility tests.
